(19) United States
(12) Patent Application Publication
(10) Pub. No.: US 2025/0082860 A1
(43) Pub. Date: Mar. 13, 2025

(54) Sub-assembly of a medicament delivery device

(71) Applicant: SHL Medical AG, Zug (CH)

(72) Inventors: Aaron Raspler, Deerfield Beach, FL (US); Nurettin Ali, Deerfield Beach, FL (US)

(21) Appl. No.: 18/727,442

(22) PCT Filed: Dec. 29, 2022
(86) PCT No.: PCT/EP2022/088038

§ 371 Date: Jul. 9, 2024

Related U.S. Application Data
(60) Provisional application No. 63/298,660, filed on Jan. 12, 2022.

(30) Foreign Application Priority Data
Jan. 24, 2022 (EP) 22152859.9

(51) Int. Cl. A61M 5/32

(52) U.S. Cl. CPC A61M 5/3202; A61M 5/326; A61M 2005/3267

(57) ABSTRACT

The present disclosure relates to a sub-assembly of a medicament delivery device comprising a syringe carrier comprising at least one first locking member, a front outer shell comprising at least one second locking member and at least one third locking member, the first locking member being arranged to be moved into locking engagement with the second locking member for locking the syringe carrier inside the front outer shell upon the syringe carrier being axially aligned with and inserted into the front outer shell, and a needle cover accommodating a needle cover spring abutting an interior front end of the needle cover in a first end, the needle cover comprising a fourth locking member and at least one cut out extending axially from a rear end of the needle cover, the needle cover being arranged to be axially aligned with and inserted into the front outer shell via a front opening of the front outer shell, the axial cut out allowing the needle cover to axially slide over the syringe carrier and have the first and second locking members, whilst in a locking engagement, move relative to the needle cover in said axial cut out until the fourth locking member of the needle cover moves into locking engagement with the third locking member of the front outer shell, wherein a second end of the needle cover spring moves into abutment with a front end of the syringe carrier.

### CROSS-REFERENCE TO RELATED APPLICATIONS

The present application is a U.S. National Phase application pursuant to 35 U.S.C. § 371 of International Application No. PCT/EP2022/088038 filed Dec. 29, 2022, which claims priority to (i) U.S. Provisional Patent Application No. 63/298,660 filed Jan. 12, 2022, and (ii) European Patent Application No. 22152859.9 filed Jan. 24, 2022. The entire disclosure contents of these applications are herewith incorporated by reference into the present application.

### TECHNICAL FIELD

The present disclosure relates to a sub-assembly of a medicament delivery device.

### BACKGROUND

Prior art medical delivery devices, such as injectors, are commonly arranged with a safety clip that must be removed by a user before medicament can be delivered to the user.

In these prior art injectors, a plunger rod is held in a position where a plunger rod spring utilized to spring-load the plunger rod is in a compressed state.

In order to release the plunger rod spring to cause the plunger rod to push medicament through an injector syringe for delivery to the user, the safety clip must first be removed which is an undesired operational step during administration of medicament. Further, this makes for a more complex assembly of the injector. There is thus room for improvement.

### SUMMARY

One objective is to solve, or at least mitigate, this problem in the art and to provide an improved sub-assembly of a medicament delivery device.

This objective is attained in an aspect by a sub-assembly of a medicament delivery device. The sub-assembly comprises a syringe carrier comprising at least one first locking member, a front outer shell comprising at least one second locking member and at least one third locking member, the first locking member being arranged to be moved into locking engagement with the second locking member for locking the syringe carrier inside the front outer shell upon the syringe carrier being axially aligned with and inserted into the front outer shell, and a needle cover accommodating a needle cover spring abutting an interior front end of the needle cover in a first end, the needle cover comprising a fourth locking member and at least one cut out extending axially from a rear end of the needle cover, the needle cover being arranged to be axially aligned with and inserted into the front outer shell via a front opening of the front outer shell, the axial cut out allowing the needle cover to axially slide over the syringe carrier and have the first and second locking members, whilst in a locking engagement, move relative to the needle cover in said axial cut out until the fourth locking member of the needle cover moves into locking engagement with the third locking member of the front outer shell, wherein a second end of the needle cover spring moves into abutment with a front end of the syringe carrier.

Advantageously, by locking the syringe carrier into the front outer shell and then locking the needle cover into the front outer shell, assembly of the medicament delivery device becomes less complex and more straightforward.

In an embodiment, a syringe support is configured to be inserted into a rear end of the syringe carrier.

In an embodiment, the syringe carrier comprises a rear section where an interior diameter of the syringe carrier decreases in a proximal direction, the decrease in diameter creating an annular surface at said rear section which the syringe support is configured to abut upon being inserted into the rear end of the syringe carrier.

In an embodiment, the first locking member of the syringe carrier comprises a radially protruding ledge and the second locking member of the front outer shell comprises an indentation with which the ledge moves into locking engagement.

In an embodiment, the second locking member of the front outer shell further comprises a radially flexible tab configured to flex outwards upon the ledge moving into contact with the radially flexible tab before moving into locking engagement with the indentation located at an end of the radially flexible tab.

In an embodiment, the third locking member of the front outer shell comprises an axially extending guide rail and the fourth locking member of the needle cover comprises an axially extending assembly track, wherein the assembly track moves into locking engagement with the guide rail upon the assembly track being axially aligned with the guide rail and the needle cover being inserted into the front outer shell.

In an embodiment, the axially extending guide rail protrudes radially from an interior of the front outer shell and the axially extending assembly track is formed by an axially extending opening in the needle cover.

In an embodiment, the axially extending guide rail protruding radially from an interior of the front outer shell has an inclining catch surface at a rear end against which a correspondingly inclining rear end of the axially extending opening in the needle cover abuts upon the needle cover being fully inserted into the front outer shell.

In an embodiment, the front outer shell comprises at least one first snap-in member at a front section, the sub-assembly further comprising a front cap comprises at least one second snap-in member configured to move into snap engagement with the first snap-in-member upon the front cap being axially moved into abutment with the front outer shell.

In an embodiment, the first snap-in member of the front outer shell comprises a radially protruding bump and the second snap-in member of the front cap comprises a first opening with which the bump moves into snap engagement.

In an embodiment, the needle cover comprises at least one third snap-in member at a front section, and the front cap comprises at least one fourth snap-in member configured to move into snap engagement with the third snap-in-member upon the front cap being axially moved into abutment with the needle cover.

In an embodiment, the third snap-in member of the needle cover comprises a radially protruding prong and the fourth snap-in member of the front cap comprises a second opening with which the prong moves into snap engagement.

In an embodiment, the syringe carrier is configured to be inserted into the front outer shell via a rear opening of the front outer shell.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated.

### BRIEF DESCRIPTION OF THE DRAWINGS

Aspects and embodiments are now described, by way of example, with reference to the accompanying drawings, in which:

FIG. 1 shows a perspective view of a prior art medicament delivery device;

FIG. 2 shows individual components of the prior art injector of FIG. 1 in an exploded view;

FIG. 3 shows a perspective view of a medicament delivery device in which a sub-assembly according embodiments may be implemented;

FIG. 4 shows individual components of the injector of FIG. 3 in an exploded view according to an embodiment;

FIGS. 5-7 show the rear wall member being pushed into the release position according to an example;

FIG. 8 illustrates a radially flexible tab according to an example;

FIGS. 9A and 9B illustrate a first step of a sub-assembly according to an embodiment;

FIGS. 10A, 10B and 10C illustrate a second step of the sub-assembly according to an embodiment;

FIG. 11 illustrates a third step of the sub-assembly according to an embodiment; and

FIG. 12 shows an illustration of the sub-assembly where a front cap is included according to an embodiment.

### DETAILED DESCRIPTION

The aspects of the present disclosure will now be described more fully hereinafter with reference to the accompanying drawings, in which certain embodiments of the invention are shown.

These aspects may, however, be embodied in many different forms and should not be construed as limiting; rather, these embodiments are provided by way of example so that this disclosure will be thorough and complete, and to fully convey the scope of all aspects of invention to those skilled in the art. Like numbers refer to like elements throughout the description.

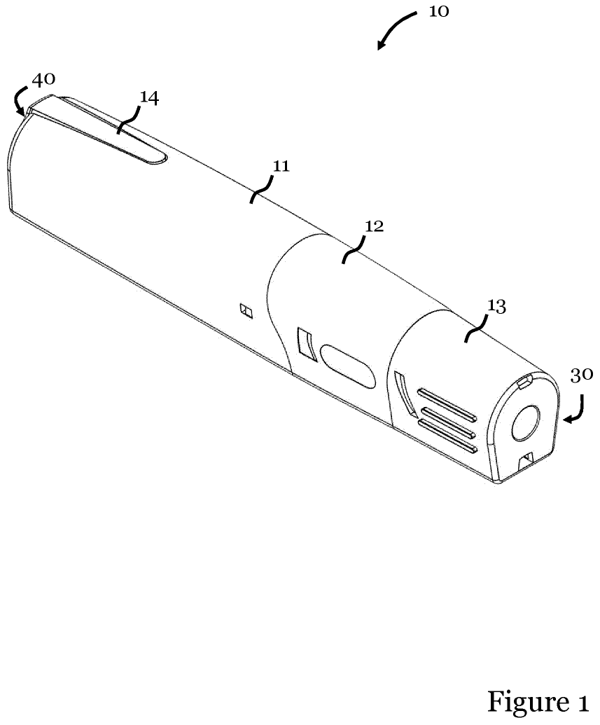

FIG. 1 shows a perspective view of a prior art medicament delivery device 10. The particular medicament delivery device 10 illustrated in FIG. 1 is an injector configured to inject a dose of medicament into the body of a user via a syringe arranged in a back outer shell 11 and front outer shell 12 of the injector 10 after a protective front cap 13 has been removed.

The prior art injector 10 is further arranged with a safety clip 14 which must be removed by the user before use of the injector 10, since a spring-loaded plunger rod arranged in the injector 10 otherwise cannot be released to cause medicament delivery.

An end of the injector 10 facing towards the user is referred to as proximal end 30 while an end of the injector 10 facing away from the user is referred to as distal end 40.

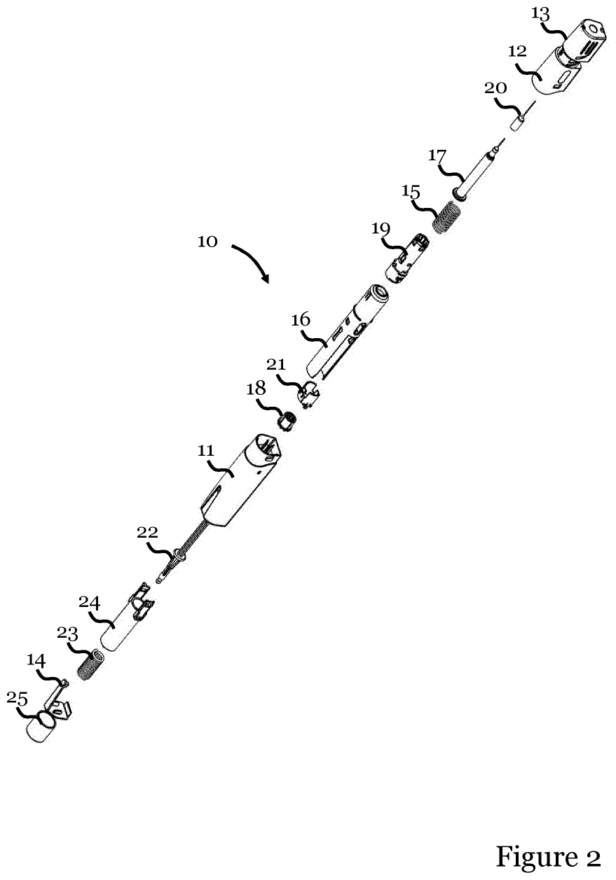

FIG. 2 shows individual components of the prior art injector 10 of FIG. 1 in an exploded view. The back outer shell 11 and the front outer shell 12 house all components except for the safety clip 14 which is attached to an exterior of the back outer shell 11, while the front cap 13 is snapped into attachment with a front end of the front outer shell 12 for protecting the user from a syringe as will be described in more detail in the following.

The injector 10 comprises a needle cover spring 15 arranged to spring-load a needle cover 16 utilized to protect the user from the syringe 17. Further associated with the syringe 17 is a syringe support 18 and a syringe carrier 19 (and medicament 20 to be delivered by the injector 10). A lock out collar 21 is employed to prevent the needle cover 16 from again being pushed towards a distal end position once medicament has been delivered and the needle cover 16 has reverted to a position at the proximal end 30 of the injector 10 for covering the syringe 17.

The injector 10 further comprises a plunger rod 22 being spring-loaded with a plunger rod spring 23 against a distal end wall of an actuator housing 24 for pushing the medicament 20 through the syringe 17 for delivery to the user upon plunger rod spring release.

A rear cap 25 is axially movable from a lock position in which the plunger rod 22 is held in a position where the plunger rod spring 23 is compressed to a release position where the rear cap 25 is moved by the needle cover 105 towards a distal end, and the plunger rod spring 23 is released, thereby causing spring release to have the plunger rod 22 push the medicament 20 through the syringe 17 for delivery. However, to cause spring release, the user must first remove the safety clip 14 which holds the rear cap 25 in the lock position when being attached to the back outer shell 11.

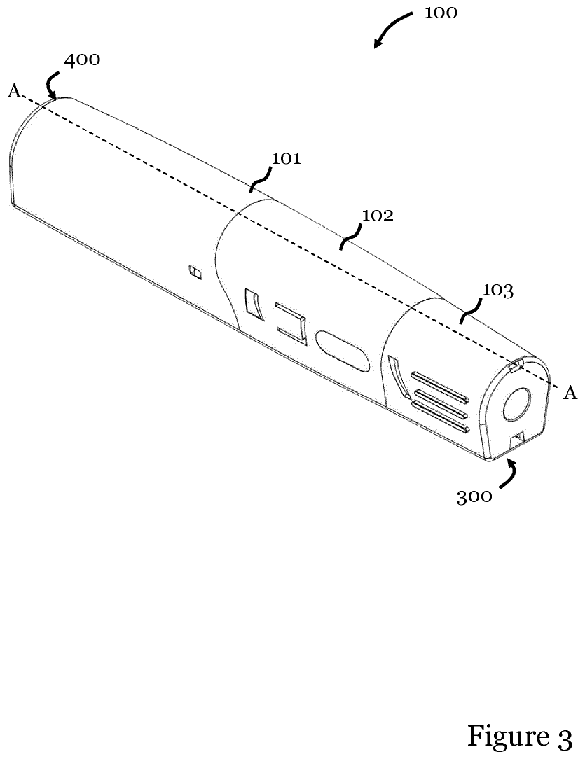

FIG. 3 shows a perspective view of a medicament delivery device 100 in which a sub-assembly according embodiments may be implemented. The particular medicament delivery device 100 illustrated in FIG. 3 is an injector configured to inject a dose of medicament into the body of a user via a syringe arranged in a back outer shell 101 and front outer shell 102 of the injector 100 after a protective front cap 103 has been removed.

An end of the injector 100 facing towards the user is referred to as proximal end 300 while an end of the injector 100 facing away from the user is referred to as distal end 400.

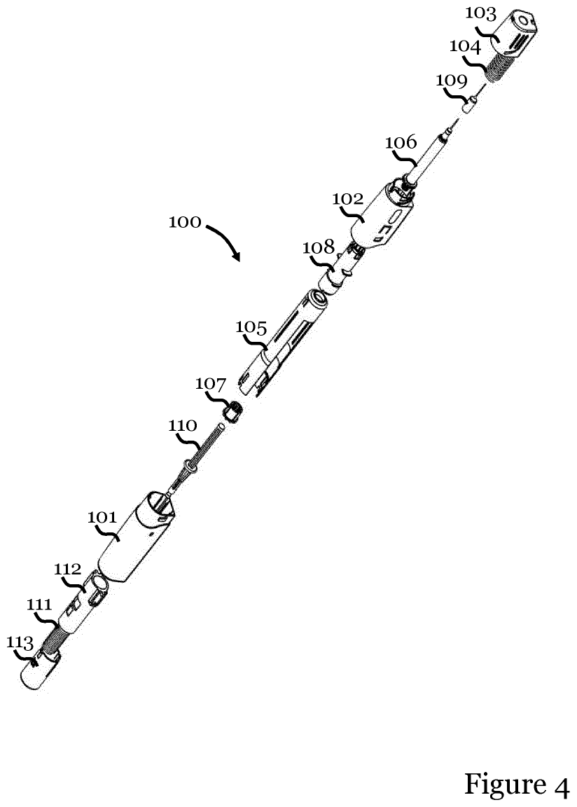

FIG. 4 shows individual components of the injector 100 of FIG. 3 in an exploded view according to a first embodiment. The back outer shell 101 and the front outer shell 102 house all components, while the front cap 103 is snapped into attachment with a front end of the front outer shell 102 for protecting the user from a syringe as will be described in more detail in the following.

The injector 100 comprises a needle cover spring 104 arranged to spring-load a needle cover 105 utilized to protect the user from the syringe 106. Further associated with the syringe 106 is a syringe support 107 and a syringe carrier 108 (and medicament 109 to be delivered by the injector 100).

The injector 100 further comprises a plunger rod 110 being spring-loaded with a plunger rod spring 111 against a distal end wall of an actuator housing 112 for pushing the medicament 109 through the syringe 106 for delivery to the user upon plunger rod spring release.

A rear cap 113 is axially movable from a lock position in which the plunger rod 110 is held in a position where the plunger rod spring 111 is fully compressed to a release position where the rear cap 113 is moved by the needle cover 105 towards a distal end, and the plunger rod spring 111 is released, thereby causing spring expansion to have the plunger rod 110 push the medicament 109 through the syringe 106 for delivery.

Thus, the rear cap 113 locks the plunger rod 111 in order to prevent accidental release of the plunger rod spring 111. Plunger rod release and subsequent medicament delivery can only be achieved upon the user removing the front cap 103 and pressing a proximal end of the needle cover 105 against a site of the body where the medicament is to be injected. The needle cover 105 will thus slide in a distal direction and push the rear cap 113 out of the lock position for plunger rod spring release.

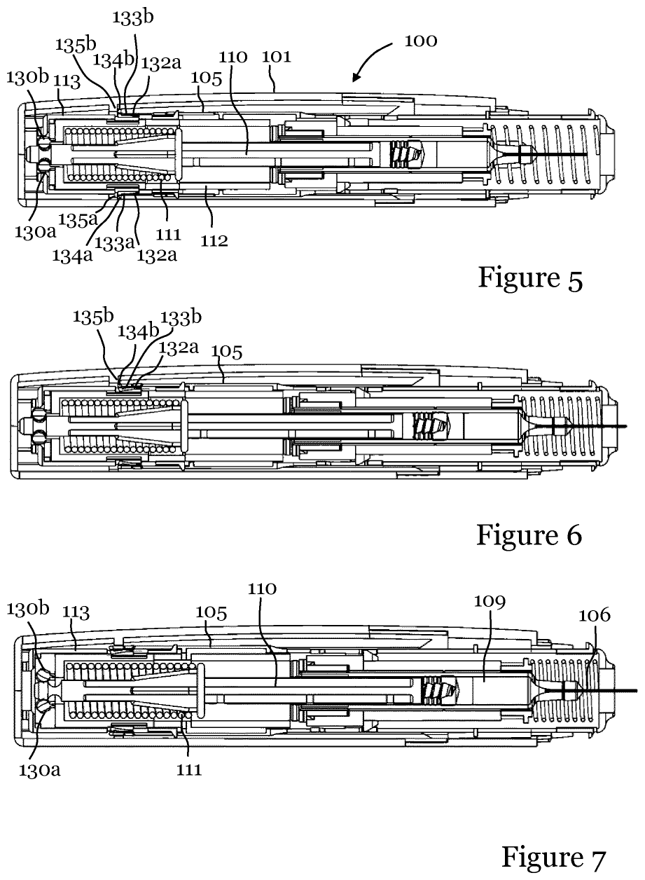

An advantage of this solution is that the rear cap 113 is held in the lock position by a radially flexible tab which holds the rear cap 113 such that the cap is axially immovable. The radially flexible tab is in this example arranged on the rear cap 113 and engages with a counter-engagement member of a device housing as will be described in FIGS. 5-8. The needle cover 105 will thus slide in a distal direction and into contact with the radially flexible tab to push the rear cap 113 out of the lock position for plunger rod spring release.

Thus, with the radially flexible tab, there is no need for the safety clip 14 of the prior art injector 10 and the user is relieved from the burden of having to remove the safety clip 14 before using the injector to deliver the medicament.

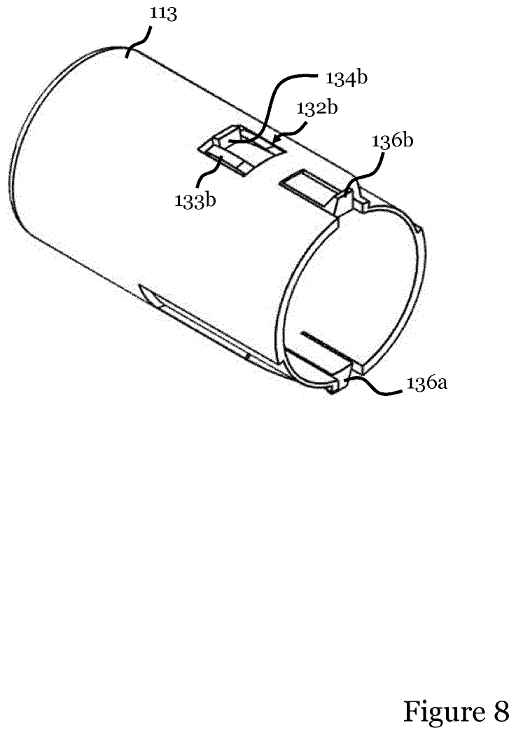

FIGS. 5-7 illustrate sectional views of the injector 100 of FIG. 3 taken along line A-A, while FIG. 8 shows the rear cap comprising the radially flexible tab in accordance with the illustration of FIG. 4. Only a few components of the exploded view of FIG. 4 will be discussed.

With reference to FIG. 5, the plunger rod 110 configured to be moveable inside the injector 100 is spring loaded by the spring 111 abutting the plunger rod 110 in one end and abutting an inner wall of the sub-assembly in another end, in this example a distal inner wall of the actuator housing 112, to compress the spring 111 upon the plunger rod 110 being moved towards a distal end of the injector 100.

Further, the sub-assembly comprises at least one arm (in this example two arms 130a, 130b) extending axially and being immovable with respect to a device housing 101 (in this example the back outer shell). The plunger rod 110 extends axially through an opening of the actuator housing 112 for contacting the arms 130a, 130b.

The arms 130a, 130b have a radially extending end section, an inner surface of which end section presses radially against the spring-loaded plunger rod 110 to axially fix the spring-loaded plunger rod 110 relative to the arms 130a, 130b.

A rear wall member embodied in this example by the rear cap 113 is axially movable from a lock position (see FIG. 5) where an inner wall of the rear cap 113 radially encloses an outer surface of each arm 130a, 130b to a release position (see FIG. 7) where the rear cap 113 is radially offset relative to the outer surface of each arm 130a, 130b.

In this example, the rear cap 113 comprises the two radially flexible tabs 132a, 132b each having an axially directed surface 134a, 134b (provided by a small hook-like member best seen in FIG. 8) adjacent to a counter surface 135a, 135b of an engagement member—in the form of two radially protruding stop members arranged at an interior of a side wall of the device housing 101—to form an axial abutment preventing the rear cap 113 from moving into the release position.

Thus, as illustrated in FIG. 5, the axially directed surface 134a, 134b of the hook-like member of each radially flexible tab 132a, 132b moves into axial abutment contact with the counter surface 135a, 135b of the respective stop member at the interior of the device housing 101, thereby holding the rear cap 113 in the lock position.

With reference in particular to FIG. 6, a release member embodied in this example by the needle cover 105 is configured to be axially moved into contact with the radial protrusions 133a, 133b of the flexible tabs 132a, 132b to radially deflect the flexible tabs 132a, 132b, thereby radially offsetting the axially directed surfaces 134a, 134b of the hook-like members from the counter surfaces 135a, 135b of the stop members of the device housing 101.

As a result, the radially flexible tabs 132a, 132b of the rear cap 113 snap out of locking engagement with the stop members of the device housing 101. The tab 132a will thus move under the stop member of the device housing 101 in a distal direction.

As illustrated in FIG. 7, upon the user further pushing the proximal end of the needle cover 105 against a site of the body for delivering the medicament, the rear cap 113 is axially moved in a distal direction from the lock position to the release position, in which the inner wall of the rear cap 113 no longer radially encloses the outer surface of each arm 130a, 130b but rather is radially offset relative to the outer surface of each arm 130a, 130b.

As a result, the arms 130a, 130b will no longer radially press against the spring-loaded plunger rod 110 in order to axially fix the spring-loaded plunger rod 110 relative to the arms 130a, 130b. The plunger rod 110 is thus released by the spring 111 expanding, and the medicament 109 is delivered via the syringe 106 to the user.

In addition to not having to use the safety clip 14 of the prior art injector 10, assembly of the injector 100 becomes less complex and more straightforward as will be discussed in the following.

The radially flexible tabs 132a, 132b described with reference to FIGS. 5-8 are discussed in further detail in U.S. application 63/290,411, which is herein incorporated by reference.

In the prior art injector 10 of FIGS. 1 and 2, during assembly the syringe carrier 19 is first locked into the needle cover 16 and thereafter the syringe carrier 19 (along with the needle cover 16 is locked into the actuator housing 24)

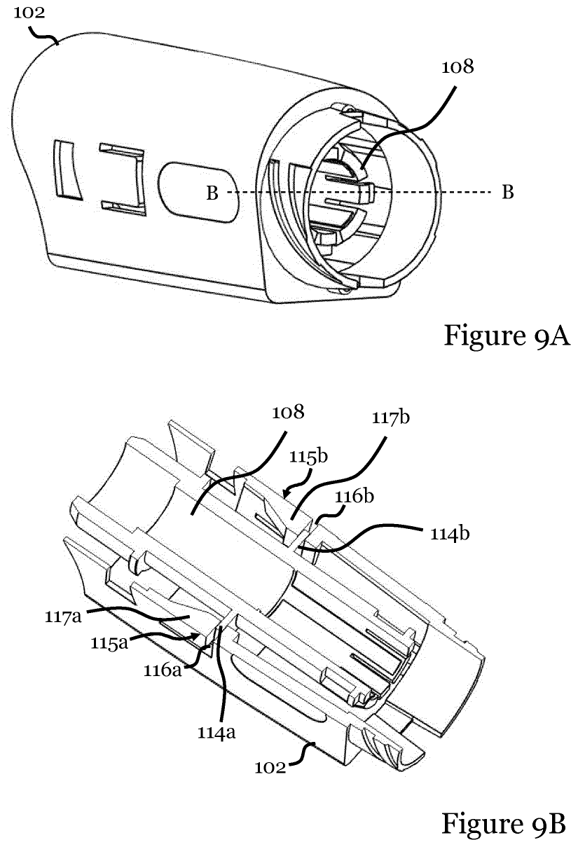

FIGS. 9A and 9B illustrate a first step of a sub-assembly according to an embodiment, where FIG. 9A shows a perspective view of the syringe carrier 108 having been inserted inside the front outer shell 102. In this example, the syringe carrier 108 has been inserted inside the front outer shell 102 via a distal opening of the front outer shell 102. FIG. 9B shows a sectional view taken along line B-B of FIG. 9A.

As can be seen in the sectional view, the syringe carrier 108 comprises at least one first locking member 114a (in this example two locking members 114a, 114b), while the front outer shell 102 comprises at least one second locking member 115a (in this example two locking members 115a, 115b).

As is understood, the first locking member 114a of the syringe carrier 108 moves into locking engagement with the second locking member 115a of the front outer shell 102 upon the syringe carrier 108 being axially aligned with and inserted into the front outer shell 102.

In this particular embodiment, the first locking member(s) 114a, 114b of the syringe carrier 108 comprises a radially protruding ledge and the second locking member(s) 115a, 115b of the front outer shell 102 comprises an at least one indentation 116a, 116b with which the ledge moves into locking engagement, but other appropriate types of locking members for locking the syringe carrier 108 to the front outer shell 102 may be envisaged.

Further in an embodiment shown in the sectional view of FIG. 9B, the second locking members 115a, 115b of the front outer shell 102 further comprises a radially flexible tab 117a, 117b configured to flex outwards upon the ledge 114a, 114b moving into contact with the radially flexible tab 117a, 117b before moving into locking engagement with the indentation 116a, 116b located at an end of the radially flexible tab 117a, 117b.

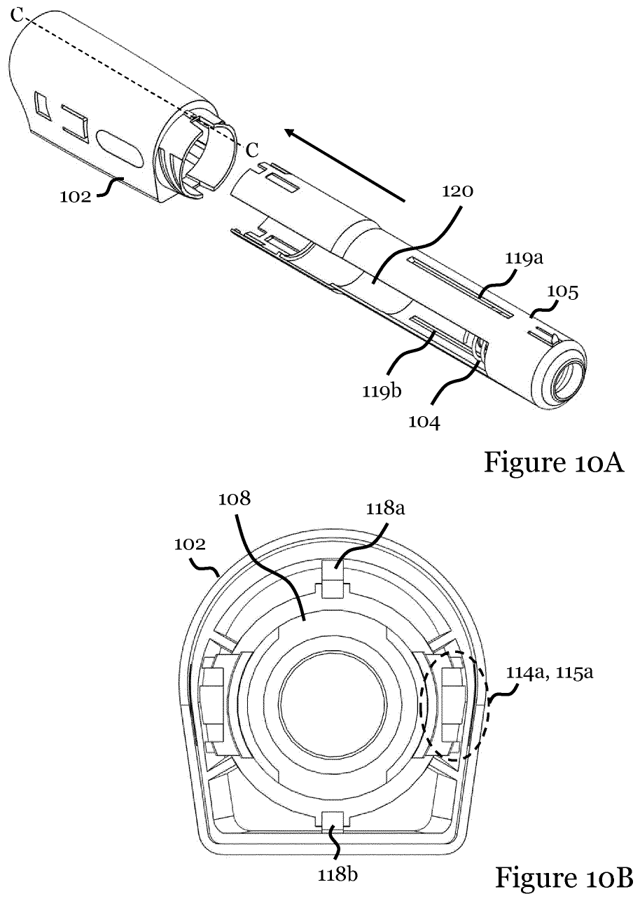

FIGS. 10A, 10B and 10C illustrate a second step of the sub-assembly according to an embodiment, where FIG. 10A shows a perspective view of the needle cover 105 (accommodating the needle cover spring 104) being inserted inside the front outer shell 102 after the syringe carrier 108 has been locked into the front outer shell 102 as was described with reference to FIGS. 9A and 9B, while FIG. 10B illustrates a rear view of the front outer shell 102, i.e. a view taken from a distal end of front outer shell 102.

FIG. 10C shows a sectional view taken along line C-C of FIG. 10A.

As shown in FIG. 10B, the front outer shell 102 comprises at least one third locking member 118a (in this example two locking members 118a, 118b), while as shown in FIG. 10A, the needle cover 105 comprises at least one fourth locking member 119a (in this example two locking members 119a, 119b), and further a cut out 120 (and in this example also a radially opposing cut out) extending axially from a rear end of the needle cover 105.

Thus, the needle cover 105 is arranged to be axially aligned with and inserted into the front outer shell 102 via a front opening. The axial cut out 120 allows the needle cover 105 to axially slide over the syringe carrier 108 and have the first and second locking members 114a, 115a (of the syringe carrier 108 and the front outer shell 102, respectively) whilst in a locking engagement move relative to the needle cover 105 in the axial cut out 120 until the fourth locking member(s) 119a, 119b of the needle cover 105 moves into locking engagement with the third locking member(s) 118a, 118b of the front outer shell 102.

As shown in the sectional view of FIG. 10C, in an embodiment, the third locking member 118a of the front outer shell 102 comprises an axially extending guide rail and the fourth locking member 119a of the needle cover 105 comprises an axially extending assembly track which moves into locking engagement with the guide rail upon the assembly track being axially aligned with the guide rail and the needle cover 105 being inserted into the front outer shell 102 and sliding over the syringe carrier 108. As is understood, a corresponding locking engagement occurs with the locking members 118b, 119b located on the radially opposite side. However, other appropriate types of locking members for locking the needle cover 105 to the front outer shell 102 may be envisaged.

In this exemplifying embodiment, the axially extending guide rail 118a protrudes radially from an interior of the front outer shell 102 and the axially extending assembly track 119a is formed by an axially extending opening in the needle cover 105.

As further shown in FIG. 10C, the movement into locking engagement will cause the needle cover spring 104 accommodated in the needle cover 105, which abuts an interior front end of the needle cover 105 in a first end, to move into abutment with a front end of the syringe carrier 108 at a second end.

Again with reference to FIG. 10C, in an embodiment, the axially extending guide rail 118a protruding radially from an interior of the front outer shell 102 has an inclining catch surface 121 at a rear end against which a correspondingly inclining rear end 122 of the axially extending opening 119a in the needle cover 105 abuts upon the needle cover 105 being fully inserted into the front outer shell 102.

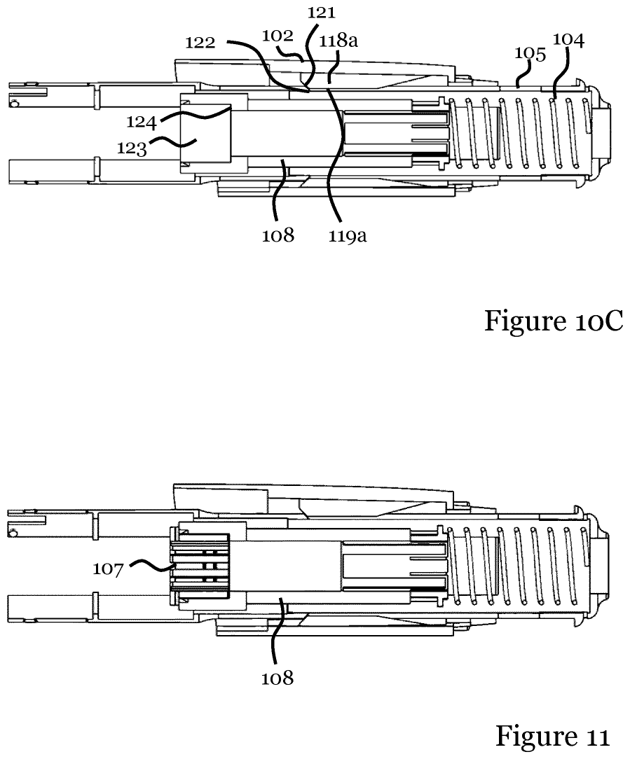

FIG. 11 illustrates a third step of the sub-assembly according to an embodiment, FIG. 11 showing a sectional view taken along line B-B of FIG. 10A.

In this embodiment, the syringe support 107 is configured to be inserted into a rear end of the syringe carrier 108, and the syringe carrier 108 comprises a rear section 123 (see FIG. 10C) where an interior diameter of the syringe carrier 108 decreases in a proximal direction, the decrease in diameter creating an annular surface 124 at said rear section 123 which the syringe support 107 is configured to abut upon being inserted into the rear end of the syringe carrier 108.

Thereafter, the sub-assembly of the above-described embodiments may be assembled with the remaining components of the injector 100 as illustrated in FIG. 4.

In a further embodiment, before or after the sub-assembly of is assembled with the remaining components of the injector 100, the front cap 103 is pushed into contact with the sub-assembly.

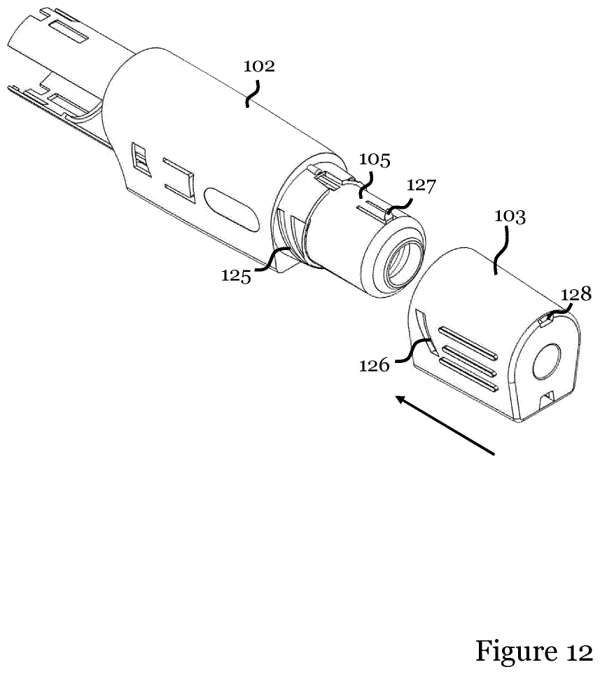

FIG. 12 shows an illustration similar to that of FIG. 10A, but further with the front cap 103 included in an embodiment.

In this embodiment, the front outer shell 102 comprises at least one first snap-in member 125 at a front section (typically one on each opposite side radially of the front section), while the front cap 103 comprises at least one second snap-in member 126 (again typically one on each opposite side radially of the front cap 103) configured to move into snap engagement with the first snap-in-member 125 upon the front cap 103 being axially moved into abutment with the front outer shell 102.

In this exemplifying embodiment, the first snap-in member 125 of the front outer shell 102 comprises a radially protruding bump and the second snap-in member 126 of the front cap 103 comprises a first opening with which the bump moves into snap engagement.

Further, in an embodiment, the needle cover 105 comprises at least one third snap-in member 127 at a front section (typically one on each side radially opposite of the front section), while the front cap 103 comprises at least one fourth snap-in member 128 configured to move into snap engagement with the third snap-in-member 127 upon the front cap 103 being axially moved into abutment with the needle cover 105.

In this exemplifying embodiment, the third snap-in member 127 of the needle cover 105 comprises a radially protruding prong and the fourth snap-in member 128 of the front cap 103 comprises a second opening with which the prong moves into snap engagement.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-la′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The aspects of the present disclosure have mainly been described above with reference to a few embodiments and examples thereof. However, as is readily appreciated by a person skilled in the art, other embodiments than the ones disclosed above are equally possible within the scope of the invention, as defined by the appended patent claims.

Thus, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-13. (canceled)

14. A sub-assembly of a medicament delivery device, comprising:
a syringe carrier comprising at least one first locking member;
a front outer shell comprising at least one second locking member and at least one third locking member, the first locking member configured to move into locking engagement with the second locking member for locking the syringe carrier inside the front outer shell upon the syringe carrier being axially aligned with and inserted into the front outer shell; and
a needle cover accommodating a needle cover spring abutting an interior front end of the needle cover in a first end, the needle cover comprising a fourth locking member and at least one cut out extending axially from a rear end of the needle cover, the needle cover being arranged to be axially aligned with and inserted into the front outer shell via a front opening of the front outer shell, the axial cut out allowing the needle cover to axially slide over the syringe carrier and have the first and second locking members, whilst in a locking engagement, move relative to the needle cover in said axial cut out until the fourth locking member of the needle cover moves into locking engagement with the third locking member of the front outer shell, wherein a second end of the needle cover spring moves into abutment with a front end of the syringe carrier.

15. The sub-assembly of claim 14, further comprising:
a syringe support configured to be inserted into a rear end of the syringe carrier.

16. The sub-assembly of claim 14, wherein:
the syringe carrier comprises a rear section where an interior diameter of the syringe carrier decreases in a proximal direction, the decrease in diameter creating an annular surface at said rear section which the syringe support is configured to abut upon being inserted into the rear end of the syringe carrier.

17. The sub-assembly of claim 14, wherein:
the first locking member of the syringe carrier comprises a radially protruding ledge and the second locking member of the front outer shell comprises an indentation with which the ledge moves into locking engagement.

18. The sub-assembly of claim 17, wherein:
the second locking member of the front outer shell further comprises a radially flexible tab configured to flex outwards upon the ledge moving into contact with the radially flexible tab before moving into locking engagement with the indentation located at an end of the radially flexible tab.

19. The sub-assembly of claim 14, wherein:
the third locking member of the front outer shell comprises an axially extending guide rail and the fourth locking member of the needle cover comprises an axially extending assembly track, wherein the assembly track moves into locking engagement with the guide rail upon the assembly track being axially aligned with the guide rail and the needle cover being inserted into the front outer shell.

20. The sub-assembly of claim 19, wherein:
the axially extending guide rail protrudes radially from an interior of the front outer shell and the axially extending assembly track is formed by an axially extending opening in the needle cover.

21. The sub-assembly of claim 20, wherein:
the axially extending guide rail protruding radially from an interior of the front outer shell has an inclining catch surface at a rear end against which a correspondingly inclining rear end of the axially extending opening in the needle cover abuts upon the needle cover being fully inserted into the front outer shell.

22. The sub-assembly of claim 14, wherein:
the front outer shell comprises at least one first snap-in member at a front section; the sub-assembly further comprising:
a front cap comprises at least one second snap-in member configured to move into snap engagement with the first snap-in-member upon the front cap being axially moved into abutment with the front outer shell.

23. The sub-assembly of claim 22, wherein:
the first snap-in member of the front outer shell comprises a radially protruding bump and the second snap-in member of the front cap comprises a first opening with which the bump moves into snap engagement.

24. The sub-assembly of claim 22, wherein:
the needle cover comprises at least one third snap-in member at a front section; and
the front cap comprises at least one fourth snap-in member configured to move into snap engagement with the third snap-in-member upon the front cap being axially moved into abutment with the needle cover.

25. The sub-assembly of claim 24, wherein:
the third snap-in member of the needle cover comprises a radially protruding prong and the fourth snap-in member of the front cap comprises a second opening with which the prong moves into snap engagement.

26. The sub-assembly of claim 14, wherein:
the syringe carrier is configured to be inserted into the front outer shell via a rear opening of the front outer shell.
